(19) United States
(12) Patent Application Publication
(10) Pub. No.: US 2022/0389482 A1
(43) Pub. Date: Dec. 8, 2022

(54) PRETREATMENT METHOD, PRETREATMENT SOLUTION, AND KIT FOR DETECTING NUCLEIC ACID OF VIRUS, AND USE THEREOF

(71) Applicant: SANSURE BIOTECH INC., Changsha (CN)

(72) Inventors: Lizhong DAI, Changsha (CN); Bozhi JI, Changsha (CN); Zhongping DENG, Changsha (CN); Jia LIU, Changsha (CN); Deyong TAN, Changsha (CN); Xu FAN, Changsha (CN)

(21) Appl. No.: 17/891,404

(22) Filed: Aug. 19, 2022

Related U.S. Application Data
(63) Continuation of application No. PCT/CN2020/090052, filed on May 13, 2020.

(30) Foreign Application Priority Data
Mar. 4, 2020 (CN) 202010143226.2

(51) Int. Cl. C12Q 1/6806; C12Q 1/6851

(52) U.S. Cl. CPC C12Q 1/6806; C12Q 1/6851; C12Q 1/706

(57) ABSTRACT

The present invention relates to the field of viral nucleic acid detection. In particular, the present invention provides a pretreatment method for viral nucleic acid detection. The method includes mixing a pretreatment solution containing a sample with a nucleic acid releasing agent and a qPCR amplification reagent, wherein the pretreatment solution includes Tris-HCl, EDTA-2Na, sodium chloride, a ribonuclease (RNase) inhibitor, and an antibiotic; and the pretreatment solution has a pH of 6.5-8.0.

### CROSS-REFERENCE TO RELATED APPLICATION

This application is a continuation application of International patent application No. PCT/CN2020/090052, filed on May 13, 2020, which claims the benefit and priority of Chinese patent application No. CN202010143226.2, filed on Mar. 4, 2020, each of which is incorporated herein by reference in its entirety and for all purposes.

### TECHNICAL FIELD

The present invention relates to the field of nucleic acid detection of viral samples, and particularly to a virus sample pretreatment method and a pretreatment solution for pretreatment of DNA/RNA virus.

### BACKGROUND

Currently, there are mainly two types of conventional virus pretreatment solutions: a Hank's matrix-based pretreatment solution and a guanidine salt-based pretreatment solution. Hank's solution is a common balanced salt solution (BSS) for virus transport. Due to various factors, the Hank's solution can preserve respiratory viruses (such as influenza virus, SARS-CoV-2, etc.) for only a few hours. After a few hours, the morphology of the virus will be affected, which may affect the efficiency of viral nucleic acid detection. After an extended time, bacteria and fungi will easily grow, resulting in a decrease in the pH value of the Hank's solution and accelerated degradation of the virus. Therefore, the Hank's solution is difficult to be used for long-term preservation and nucleic acid detection of viruses. The concentration of guanidine hydrochloride or Rnasin in the guanidine salt-based virus pretreatment solution is relatively high (3 M to 5 M), and a high concentration of a guanidine salt is suitable for virus inactivation at room temperature. However, the high concentration of the guanidine salt greatly inhibits nucleic acid extraction or purification (such as based on magnetic beads), and thus it needs to be washed multiple times or used in a spin column-based method.

The extraction free nucleic acid release and amplification technology (EFNART), simply referred to as the “one-step” technique, refers to directly performing a nucleic acid amplification test for a sample by directly combining a sample nucleic acid releasing agent with a strong alkaline property and a highly compatible amplification system in a circumstance where no nucleic acid extraction or purification of the sample is required. The “one-step method” will greatly reduce time for viral nucleic acid detection, particularly for RNA viruses. The method is expected to save time by 60% or more and improve detection efficiency by 50% in comparison with a traditional amplification method performed after nucleic acid extraction.

In the event of a major epidemic (for example, the SARS-CoV-2 epidemic that occurred from 2019 to 2020), detection based on the one-step method is preferred because it can save detection time and improve efficiency, and will make a huge contribution to epidemic control.

However, existing common virus pretreatment solutions, such as the Hank's pretreatment solution and the guanidine salt-based pretreatment solution, cannot be well used for subsequent detection based on the one-step method. Thus, it is difficult to apply the existing common virus pretreatment solutions to scenarios in which rapid detection and screening of virus samples are required.

Therefore, there is a need in the art for a sample pretreatment solution that can be adequate for use in one-step method-based viral nucleic acid detection.

### SUMMARY

In view of this, in a first aspect, the present invention provides a pretreatment method for viral nucleic acid detection, the method including: mixing a sample preserved in a pretreatment solution with a nucleic acid releasing agent and a qPCR amplification reagent;

wherein the pretreatment solution contains: Tris-HCl, EDTA-2Na, sodium chloride, a ribonuclease (RNase) inhibitor, and an antibiotic; and

the pretreatment solution has a pH of 6.5 to 8.0.

In the present invention, the Tris-HCl may be present at a concentration of about 10 mM to about 200 mM, preferably at a concentration of about 80 mM to about 120 mM, and most preferably at a concentration of about 100 mM.

In the present invention, the EDTA-2Na may be present at a concentration of about 8 mM to about 50 mM, preferably at a concentration of about 10 mM to about 15 mM, and most preferably at a concentration of about 10 mM.

In the present invention, the sodium chloride may be present at a concentration of about 0.5% (w/v) to about 2% (w/v), preferably at a concentration of about 0.8% (w/v) to about 1% (w/v), and most preferably at a concentration of about 0.9% (w/v).

In the present invention, the term “ribonuclease (RNase) inhibitor” refers to a chemical substance that can inhibit and thus inactivate RNase, including, but is not limited to, diethyl pyrocarbonate (DEPC), a RNase protein inhibitor (RNasin), ribonucleoside vanadyl complexes, SDS, etc.

In the present invention, the RNase inhibitor may be present at a concentration of about 2 U/mL to about 800 U/mL, for example, about 40 U/mL, about 50 U/mL, and about 100 U/mL; preferably at a concentration of about 10 U/mL to about 30 U/mL; and most preferably at a concentration of 20 U/mL.

In the present invention, the pH value ranges from 6.5 to 8.0, preferably from 7.0 to 8.0, and is most preferably 7.5.

The antibiotic includes, but is not limited to, Proclin antibiotics (such as Proclin 300 and Proclin 950) and NaN₃.

For example, in a case where Proclin 300 is used as the antibiotic, the concentration of Proclin 300 may be about 0.01% (v/v). For another example, in a case where Proclin 950 is used as the antibiotic, the concentration of Proclin 950 may be about 0.04% (v/v), but the present invention is not limited thereto.

In a specific embodiment, the pretreatment solution contains Tris-HCl at a concentration of 100 mM, EDTA-2Na at a concentration of 10 mM, sodium chloride at a concentration of 0.9% (w/v), the RNase inhibitor at a concentration of 20 U/mL, and Proclin 950 at a concentration of 0.04% (v/v),

and the aforementioned pretreatment solution is adjusted to a pH value of 7.5.

As used herein, the term “sample” refers to a sample that may contain a virus. The sample may be derived from human or animal blood, feces, urine, oral epithelial cells, exfoliated cells, buccal swabs, throat swabs, etc.

The term “pretreatment” mentioned in the present invention refers to treatment prior to performing a test for a sample, particularly treatment prior to nucleic acid detection of the sample (based on the one-step method).

The “pretreatment solution” mentioned in the present invention refers to a liquid for pretreating a virus sample.

In the present invention, the virus may be a DNA virus or an RNA virus.

In a preferred embodiment, the virus is an RNA virus.

In a more preferred embodiment, the virus is coronavirus (such as SARS-CoV-2), respiratory syncytial virus, and enterovirus.

A nucleic acid detection reaction solution prepared by the pretreatment method of the present invention can be used to directly perform qPCR, without requiring an extraction or purification process, thereby improving detection efficiency and reducing detection time. The antibiotic and the RNase inhibitor employed in the present invention can be used for pretreatment of DNA viruses or RNA viruses, and can prevent the activity of a preserved virus from being affected by various microorganisms, such as bacteria, growing at room temperature. The RNA virus is more easily degraded due to the ubiquitous RNase. Damage to RNA that is caused by samples (various sample types such as oral epithelial cells, exfoliated cells, throat swabs, etc.) or sampling consumables in a medical sampling process is avoided while the method of the present invention is used, which is very conducive to long-term preservation and detection of the RNA virus.

It should be noted that in a case where the sample treated with the pretreatment solution of the present invention is not preserved but directly (i.e., 0 hr after treatment) subjected to EFNART-based detection, as confirmed in the examples below, components of the pretreatment solution of the present invention have an effect of enhancing RT-PCT.

In a second aspect, the present invention provides a method of detecting viral nucleic acid in a sample, including: directly performing detection by using a reaction solution prepared by the aforementioned method to perform qPCR amplification.

A sample releasing agent refers to a chemical agent that can release nucleic acid in a sample, such as a chemical agent with strong acidity or strong basicity. An exemplary sample releasing agent may include one or more of components such as 0.01-0.5 mmol/L surfactin, 100-200 mmol/L potassium chloride, 50-200 mmol/L lithium chloride, triethanolamine dodecyl sulfate with a mass/volume ratio of 0.1-1%, ethyl phenyl polyethylene glycol (NP-40) with a volume/volume ratio of 0.1-1%, sodium dodecyl sulfonate with a mass/volume ratio of 0.01-2%, ethanol with a volume/volume ratio of 0.05-1%, etc., but the present invention is not limited thereto.

A qPCR amplification reagent refers to a reagent for a real-time quantitative nucleic acid amplification test. It can be understood by those skilled in the art that the qPCR amplification reagent usually contains DNA polymerase, dNTP, a PCR buffer solution, etc. For example, when RNA detection is to be performed, reverse transcriptase may also be further included. It is not difficult to understand that those skilled in the art can determine the components and concentrations of PCR reaction reagents according to specific needs (for example, the type and the content of a virus, etc.).

The term “one-step method” mentioned in the present invention refers to the extraction free nucleic acid release and amplification technology (EFNART). The EFNART refers to directly performing a nucleic acid amplification test for a sample by directly combining a sample nucleic acid releasing agent with strong basicity and a highly compatible amplification system in a circumstance where no nucleic acid extraction or purification of the sample is required.

When detection of viral nucleic acid in the sample is performed based on the one-step method, the sample in the preservation solution or pretreatment solution, the sample releasing agent, and the qPCR reaction solution are directly amplified after being mixed. Mixing may be performed at a general ratio in the art. In an exemplary embodiment, the sample in the preservation solution or the pretreatment solution, the sample releasing agent, and the qPCR reaction solution may be present in the ratio of about 5:5:40, about 10:10:30, or about 5:15:30 (v/v), but the present invention is not limited thereto.

In a third aspect, the present invention provides a pretreatment solution for viral nucleic acid detection, including:

Tris-HCl, EDTA-2Na, sodium chloride, an RNase inhibitor, and an antibiotic; and

the pretreatment solution has a pH of 6.5 to 8.0.

In the present invention, Tris-HCl may be present at a concentration of about 10 mM to about 200 mM, preferably at a concentration of about 80 mM to about 120 mM, and most preferably at a concentration of about 100 mM.

In the present invention, EDTA-2Na may be present at a concentration of about 8 mM to about 50 mM, preferably at a concentration of about 10 mM to about 15 mM, and most preferably at a concentration of about 10 mM.

In the present invention, sodium chloride may be present at a concentration of about 0.5% (w/v) to about 2% (w/v), preferably at a concentration of about 0.8% (w/v) to about 1% (w/v), and most preferably at a concentration of about 0.9% (w/v).

In the present invention, the RNase inhibitor may be present at a concentration of about 2 U/mL to about 800 U/mL, preferably at a concentration of about 10 U/mL to about 30 U/mL, and most preferably at a concentration of 20 U/mL.

In the present invention, the pH value ranges from 6.5 to 8.0, preferably from 7.0 to 8.0, and is most preferably 7.5.

The antibiotic includes, but is not limited to, Proclin antibiotics (such as Proclin 300 and Proclin 950) and NaN₃.

For example, in a case where Proclin 300 is used as the antibiotic, the concentration of Proclin 300 may be about 0.01% (v/v). For another example, in a case where Proclin 950 is used as the antibiotic, the concentration of Proclin 950 may be about 0.04% (v/v), but the present invention is not limited thereto.

In a specific embodiment, the pretreatment solution includes Tris-HCl at a concentration of 100 mM, EDTA-2Na at a concentration of 10 mM, sodium chloride at a concentration of 0.9% (w/v), the RNase inhibitor at a concentration of 20 U/mL, and Proclin 950 at a concentration of 0.04% (v/v);

and the aforementioned pretreatment solution is adjusted to a pH value of 7.5.

In some specific embodiments, the virus may be a DNA virus or an RNA virus.

In a preferred embodiment, the virus is an RNA virus.

In a more preferred embodiment, the virus is coronavirus (such as SARS-CoV-2), respiratory syncytial virus, and enterovirus.

In a fourth aspect, the present invention provides use of the pretreatment solution in preparation of a one-step method-based nucleic acid amplification detection kit for virus detection.

In a fifth aspect, the present invention provides a kit for viral nucleic acid detection based on one-step method, the kit including the aforementioned pretreatment solution.

Further, the kit further includes a sample releasing agent and a qPCR amplification reagent.

### BRIEF DESCRIPTION OF THE DRAWINGS

FIG. 1 is a graph showing the results of “one-step method” testing of the SARS-CoV-2 nucleic acid in the samples gradient-diluted by using the pretreatment method of the present invention and after being preserved at room temperature for 72 hr.

### DETAILED DESCRIPTION

The present invention will be described in detail below in conjunction with specific embodiments and examples, and the advantages and various effects of the present invention will be more clearly presented therefrom. It should be understood by those skilled in the art that these specific embodiments and examples are used to illustrate the present invention, but not to limit the present invention.

### Example 1. Pretreatment and Rapid Detection of Respiratory Syncytial Virus (RSV) Throat Swab Samples with the Present Invention

In order to evaluate the virus pretreatment solution of the present invention, comparative analysis was performed on the virus pretreatment solution of the present invention (Tris-HCl at a concentration of 100 mM, EDTA-2Na at a concentration of 10 mM, sodium chloride at a concentration of 0.9% (w/v), RNasin at a concentration of 20 U/mL, and Proclin 950 at a concentration of 0.04% (v/v)), a saline solution, and commercially available virus pretreatment solutions. The comparison method was employed by performing dilution (1:9, v/v) pretreatment on clinically diagnosed positive RSV throat swab samples, and direct amplification of the samples at 0 hr, 24 hr, 48 hr, and 72 hr, respectively at room temperature (25° C.). The detection efficiency of real-time quantitative PCR (real-time qPCR) under the room temperature pretreatment condition was compared by Ct values to evaluate the effects of different pretreatment solutions on virus pretreatment. The qPCR amplification test was employed by using the EFNART “one-step method” technique, such that a real-time qPCR amplification test was directly performed in a PCR amplification tube at a ratio of a pretreatment solution with the sample:a nucleic acid releasing agent:a PCR amplification reagent of 10:10:30.

Real-time qPCR amplification testing procedures are as shown in Table 1, and the results are as shown in Table 2.

TABLE 1

| | | | Number |
|---|---|---|---|
| Step | Temperature | Time | of Cycles |
| Reverse transcription | 60° C. | 30 min | 1 |
| Pre-denaturation | 95° C. | 1 min | 1 |
| Denaturation | 95° C. | 15 sec | 40-45 |
| Annealing, extension, and | 60° C. | 30 sec | |
| fluorescence collection | | | |

TABLE 2

| | Ct Value by Detection in | | | | Ct Value by Detection in | | | |
|---|---|---|---|---|---|---|---|---|
| | Original Sample | | | | 10-fold Diluted Sample | | | |
| Pretreatment Time | 0 hr | 24 hr | 48 hr | 72 hr | 0 hr | 24 hr | 48 hr | 72 hr |
| Pretreatment solution | 30.69 | 31.05 | 30.98 | 31.24 | 33.56 | 33.98 | 33.78 | 34.01 |
| of the present | | | | | | | | |
| invention | | | | | | | | |
| Saline solution | 31.04 | 32.15 | 33.24 | 34.65 | 34.12 | 35.26 | 36.42 | 36.98 |
| Hank's pretreatment | 35.12 | 38.20 | No Ct | No Ct | No Ct | No Ct | No Ct | No Ct |
| solution | | | | | | | | |
| Guanidine | No Ct | No Ct | No Ct | No Ct | No Ct | No Ct | No Ct | No Ct |
| hydrochloride | | | | | | | | |
| pretreatment solution | | | | | | | | |

The aforementioned results show that with the virus pretreatment method of the present invention, pretreatment at room temperature for 24 hr, the gradient-diluted RSV sample can be well pretreatment, and can be directly used for EFNART detection, and with the pretreatment method based on saline solution matrix, after pretreatment for a long time, the Ct values is delayed, and the nucleic acid of the virus is degraded to a certain extent, which affects the amplification efficiency. In contrast, the Hank's pretreatment solution and the guanidine salt pretreatment solution, which are commonly used and commercially available, cannot provide an effective way of performing virus pretreatment and a PCR amplification test in this scheme.

### Example 2. Nucleic Acid Pretreatment and Rapid Detection SARS-CoV-2 Samples after Purification with the Present Invention

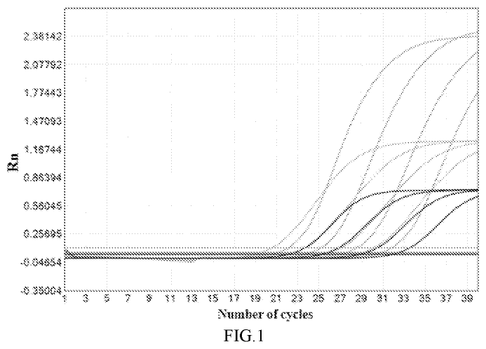

In order to evaluate the virus pretreatment solution of the present invention, comparative analysis was performed on the virus pretreatment solution of the present invention (Tris-HCl at a concentration of 100 mM, EDTA-2Na at a concentration of 10 mM, sodium chloride at a concentration of 0.9% (w/v), RNasin at a concentration of 20 U/mL, and Proclin 300 at a concentration of 0.01% (v/v)), a saline solution, and commercially available virus pretreatment solutions. The comparison method was employed by performing dilution (1:9, v/v) pretreatment on clinically diagnosed positive SARS-CoV-2 nucleic acid, and direct amplification of the sample at 0 hr, 24 hr, 48 hr, and 72 hr, respectively at room temperature (25° C.). The detection efficiency of real-time qPCR under the room temperature pretreatment condition was compared by Ct values to evaluate the effects of different pretreatment solutions on virus pretreatment. The qPCR amplification test was employed by using the EFNART “one-step method” technique, such that a real-time qPCR amplification test was directly performed in a PCR amplification tube at a ratio of a pretreatment solution with the nucleic acid:a nucleic acid releasing agent:a PCR amplification reagent of 10:10:30. The results are as shown in Table 3. The SARS-CoV-2 nucleic acid gradient-diluted by using the virus pretreatment method of the present invention was preserved at room temperature for 72 hr. The results of the “one-step method” testing are as shown in FIG. 1.

TABLE 3

| | Ct Value by Detection in | | | | Ct Value by Detection in | | | |
|---|---|---|---|---|---|---|---|---|
| | Original Sample | | | | 10-fold Diluted Sample | | | |
| Pretreatment Time | 0 hr | 24 hr | 48 hr | 72 hr | 0 hr | 24 hr | 48 hr | 72 hr |
| Pretreatment solution | 28.59 | 28.68 | 29.01 | 28.97 | 32.01 | 31.89 | 32.14 | 32.20 |
| of the present | | | | | | | | |
| invention | | | | | | | | |
| Saline solution | 28.95 | 29.48 | 29.98 | 30.54 | 33.01 | 3.21 | 33.14 | 34.28 |
| Hank's pretreatment | 31.02 | 32.35 | 33.62 | No Ct | No Ct | No Ct | No Ct | No Ct |
| solution | | | | | | | | |
| Guanidine | No Ct | No Ct | No Ct | No Ct | No Ct | No Ct | No Ct | No Ct |
| hydrochloride | | | | | | | | |
| pretreatment solution | | | | | | | | |

The aforementioned results show that with the virus pretreatment method of the present invention, pretreatment at room temperature for 24 hr, the gradient-diluted SARS-CoV-2 nucleic acid can be well pretreated, and can be directly used for EFNART detection, and with the pretreatment method based on saline solution matrix, after pretreatment for a long time, the Ct values is delayed, and the nucleic acid of the virus is degraded to a certain extent, which affects the amplification efficiency. In contrast, the Hank's pretreatment solution and the guanidine salt pretreatment solution, which are commonly used and commercially available, cannot provide an effective way of performing virus pretreatment and a PCR amplification test in this scheme.

### Example 3. Pretreatment and Rapid Detection of Enterovirus (EV) Universal Throat Swab Samples with the Present Invention

In order to evaluate the virus pretreatment solution of the present invention, comparative analysis was performed on the virus pretreatment solution of the present invention (Tris-HCl at a concentration of 100 mM, EDTA-2Na at a concentration of 10 mM, sodium chloride at a concentration of 0.9% (w/v), SDS at a concentration of 0.1%, and Proclin 950 at a concentration of 0.04% (v/v)), a saline solution, and commercially available virus pretreatment solutions. The comparison method was employed by performing dilution (1:9, v/v) pretreatment on clinically diagnosed positive EV universal throat swab samples, and direct amplification of the samples at 0 hr, 24 hr, 48 hr, and 72 hr, respectively at room temperature (25° C.). The detection efficiency of real-time qPCR under the room temperature pretreatment condition was compared by Ct values to evaluate the effects of different pretreatment solutions on virus pretreatment. The qPCR amplification test was employed by using the EFNART “one-step method” technique, such that a real-time qPCR amplification test was directly performed in a PCR amplification tube at a ratio of a pretreatment solution with the sample:a nucleic acid releasing agent:a PCR amplification reagent of 10:10:30. The results are as shown in Table 4:

TABLE 4

| | Ct Value by Detection in | | | | Ct Value by Detection in | | | |
|---|---|---|---|---|---|---|---|---|
| | Original Sample | | | | 10-fold Diluted Sample | | | |
| Pretreatment Time | 0 hr | 24 hr | 48 hr | 72 hr | 0 hr | 24 hr | 48 hr | 72 hr |
| Pretreatment solution | 26.12 | 26.32 | 26.25 | 26.34 | 29.65 | 29.78 | 29.82 | 29.16 |
| of the present | | | | | | | | |
| invention | | | | | | | | |
| Saline solution | 27.32 | 27.15 | 28.64 | 29.01 | 30.98 | 31.65 | 32.27 | 32.59 |
| Hank's pretreatment | 30.12 | 30.54 | 33.28 | 33.42 | No Ct | No Ct | No Ct | No Ct |
| solution | | | | | | | | |
| Guanidine | No Ct | No Ct | No Ct | No Ct | No Ct | No Ct | No Ct | No Ct |
| hydrochloride | | | | | | | | |
| pretreatment solution | | | | | | | | |

The aforementioned results show that with the virus pretreatment method of the present invention, pretreatment at room temperature for 24 hr, the gradient-diluted EV samples can be well pretreated, and can be directly used for EFNART detection, and with the pretreatment method based on saline solution matrix, after pretreatment for a long time, the Ct values is delayed, and the nucleic acid of the virus is degraded to a certain extent, which affects the amplification efficiency. In contrast, the Hank's pretreatment solution and the guanidine salt pretreatment solution, which are commonly used and commercially available, cannot provide an effective way of performing virus pretreatment and a PCR amplification test in this scheme.

### Example 4. Pretreatment Capacity of Virus Pretreatment Solution of the Present Invention for RNA Virus

Compared with DNA viruses, pretreatment and detection of RNA viruses are more susceptible to environmental factors due to higher requirements. A pretreatment process involving RNase contained in consumables has a particularly vital impact on detection of RNA viruses. In order to evaluate the pretreatment effect of the virus pretreatment solution of the present invention (Tris-HCl at a concentration of 100 mM, EDTA-2Na at a concentration of 10 mM, sodium chloride at a concentration of 0.9% (w/v), Rnasin at a concentration of 20 U/mL, and Proclin 950 at a concentration of 0.04% (v/v)) on RNA viruses, nucleic acid extracted from an SARS-CoV-2 sample pretreated with the virus pretreatment solution of the present invention was divided into two parts (A/B), at the same time, nucleic acid (C) extracted from an SARS-CoV-2 sample with the same concentration that was pretreated in the saline solution was prepared, 0.25 μg/mL RNase A was respectively added into solution A and solution C, the three solutions containing the SARS-CoV-2 nucleic acid were pretreated at room temperature (25° C.) for 24 hr, and then the RNA virus was pretreated and detected with the pretreatment solution of the present invention by adopting the EFNART “one-step method” technique. Detection was conducted by directly performing a real-time qPCR amplification test in a PCR amplification tube at a ratio of a pretreatment solution with the nucleic acid:a nucleic acid releasing agent:a PCR amplification reagent of 10:10:30. The results are as shown in Table 5.

TABLE 5

| SARS-CoV-2 Nucleic Acid | | | |
|---|---|---|---|
| Pretreatment Condition | Ct Value by Detection | | |
| Pretreated nucleic acid sample A | FAM | HEX | ROX |
| (pretreatment solution of the present | 30.8 | 26.8 | 29.6 |
| invention containing 0.25 g/mL Rnase A) | | | |
| Pretreated nucleic acid sample B | FAM | HEX | ROX |
| (pretreatment solution of the present | 30.9 | 27.1 | 30.2 |
| invention without 0.25 g/mL Rnase A) | | | |
| Pretreated nucleic acid sample C | FAM | HEX | ROX |
| (pretreatment with saline solution | No Ct | 27.1 | No Ct |
| containing 0.25 g/mL Rnase A) | | | |
| Pretreated nucleic acid sample D | FAM | HEX | ROX |
| (pretreatment with saline solution | 32.2 | 29.4 | 32.9 |
| without 0.25 g/mL Rnase A) | | | |

It is proved by the experiments that in a case where pretreated nucleic acid sample D is taken as a reference, the addition of 0.25 μg/mL RNase A in the present invention has no influence on nucleic acid detection effect of SARS-CoV-2, and effective components in the present invention can digest RNase, reduce the influence of RNase on experimental detection, and can ensure the efficiency of direct detection of RNA viruses. It can be seen from pretreated nucleic acid sample C, the addition of 0.25 μg/mL RNase A included in the experimental conditions can digest and degrade the RNA in the experiment, thereby greatly affecting direct detection of RNA viruses and leading to risk of missed detection.

### Example 5. Pretreatment and Rapid Detection of Hepatitis B Virus (HBV) Serum Sample by Using Virus Pretreatment Solution of the Present Invention

In order to evaluate the effect of the virus pretreatment solution of the present invention on DNA virus preservation and amplification detection, comparative analysis was performed on the virus pretreatment solution of the present invention (Tris-HCl at a concentration of 100 mM, EDTA-2Na at a concentration of 10 mM, sodium chloride at a concentration of 0.9% (w/v), RNasin at a concentration of 20 U/mL, and Proclin 950 at a concentration of 0.04% (v/v)), a saline solution, and commercially available virus pretreatment solutions. The comparison method was employed by performing dilution (1:9, v/v) pretreatment on clinically diagnosed positive HBV serum samples, and direct amplification of the samples at 0 hr, 24 hr, 48 hr, and 72 hr, respectively at room temperature (25° C.). The detection efficiency of real-time qPCR under the room temperature pretreatment condition was compared by Ct values to evaluate the effects of different pretreatment solutions on virus pretreatment. The qPCR amplification test was employed by using the EFNART “one-step method” technique, such that a real-time qPCR amplification test was directly performed in a PCR amplification tube at a ratio of a pretreatment solution with the DNA virus:a nucleic acid releasing agent:a PCR amplification reagent of 10:10:30. The results are as shown in Table 6.

TABLE 6

| | Ct Value by Detection in HBV Serum Sample | | | |
|---|---|---|---|---|
| Pretreatment Time | 0 hr | 24 hr | 48 hr | 72 hr |
| Pretreatment solution | 32.23 | 31.98 | 32.24 | 32.67 |
| of the present invention | | | | |
| Saline solution | 33.43 | 33.18 | 33.54 | 33.87 |
| Hank’s pretreatment | 33.74 | 33.49 | 33.85 | 34.18 |
| solution | | | | |
| Guanidine hydrochloride | No Ct | No Ct | No Ct | No Ct |
| pretreatment solution | | | | |

The aforementioned results show that although the original design of the virus pretreatment method of the present invention was aimed at virus preservation matrix for RNA viruses for performing amplification based on the “one-step method”, the virus pretreatment method can also be used for preservation and detection of DNA viruses by performing amplification based on the one-step method.

### Example 6. Verification of Effectiveness of Various Components of Virus Pretreatment Solution of the Present Invention

In order to verify the effectiveness of various components in the present invention, after relevant components in the present invention were adjusted and reduced, the sample was preserved and was subjected to pretreatment and a comparison experiment. Separately optimized components included an RNase inhibitor, an antibiotic, EDTA-2Na, etc. The adjusted concentrations of various components are as shown in Table 7. qPCR amplification testing was directly performed after RSV was preserved with the pretreatment solutions at room temperature for 24 hr that were prepared at different concentrations, and the detection efficiency of real-time qPCR under the room temperature pretreatment condition was compared by Ct values to evaluate the effects of different pretreatment solutions on virus pretreatment. The qPCR amplification test was employed by using the EFNART “one-step method” technique, such that a real-time qPCR amplification test was directly performed in a PCR amplification tube at a ratio of a pretreatment solution with the sample:a nucleic acid releasing agent:a PCR amplification reagent of 10:10:30.

TABLE 7
Detection Effects of Separately Optimized components
in the Present Invention on Pretreatment

| | | Antibiotic | | | | Solution pH Value | |
|---|---|---|---|---|---|---|---|
| RNase inhibitor | | (Proclin 950) | | EDTA-2Na | | Adjustment | |
| Concentration | Ct value | Concentration | Ct value | Concentration | Ct value | pH value | Ct value |
| 0 | No Ct | 0 | No Ct | 0 | 38.2 | 4 | 38.54 |
| 2 U/mL | 31.54 | 0.01% | 31.48 | 10 mM | 31.4 | 6.5 | 31.64 |
| 40 U/mL | 32.4 | 0.04% | 32.4 | 50 mM | 33.4 | 8 | 31.81 |
| 8000 U/mL | 35.42 | 1% | 37.62 | 200 mM | No Ct | 12 | No Ct |

Based on the aforementioned experiments, the chemical components in the present invention are essential components, and at the concentrations in the present invention, the pretreatment and detection of RNA viruses by employing one-step RT-PCR are optimal.

1. A pretreatment method for viral nucleic acid detection, the method comprising: mixing a sample preserved in a pretreatment solution with a nucleic acid releasing agent and a qPCR amplification reagent, wherein the pretreatment solution comprises Tris-HCl, EDTA-2Na, sodium chloride, a ribonuclease (RNase) inhibitor, and an antibiotic; and the pretreatment solution has a pH of 6.5-8.0.

2. The method according to claim 1, wherein in the pretreatment solution, the Tris-HCl is present at a concentration of 10 mM to 200 mM, the EDTA-2Na is present at a concentration of 8 mM to 50 mM, the sodium chloride is present at a concentration of 0.5% to 2% (w/v), the RNase inhibitor is present at a concentration of 2 U/mL to 800 U/mL, and the antibiotic is present at a concentration of 0.005% to 0.05%.

3. The method according to claim 1, wherein in the pretreatment solution, the Tris-HCl is present at a concentration of 80 mM to 120 mM, the EDTA-2Na is present at a concentration of 10 mM to 15 mM, the sodium chloride is present at a concentration of 0.8% to 1% (w/v), and the RNase inhibitor is present at a concentration of 10 U/mL to 30 U/mL.

4. The method according to claim 1, wherein the RNase inhibitor is diethyl pyrocarbonate (DEPC), a RNase protein inhibitor (RNasin), a ribonucleoside vanadyl complex, or SDS; and/or, the antibiotic is a Proclin antibiotic or NaN₃.

5. The method according to claim 1, wherein the antibiotic is Proclin 300 or Proclin 950.

6. The method according to claim 1, wherein a reaction solution prepared by the method can be used to directly perform a qPCR amplification test.

7. The method according to claim 1, wherein the virus is a DNA virus or an RNA virus.

8. The method according to claim 1, wherein the virus is coronavirus, respiratory syncytial virus, or enterovirus.

9. The method according to claim 1, wherein the virus is SARS-CoV-2.

10. The method according to claim 1, wherein the sample is derived from human or animal blood, feces, urine, oral epithelial cells, exfoliated cells, buccal swabs, or throat swabs.

11. A pretreatment solution for viral nucleic acid detection, the pretreatment solution comprising: Tris-HCl, EDTA-2Na, sodium chloride, a ribonuclease (RNase) inhibitor, and an antibiotic, wherein the pretreatment solution has a pH of 6.5-8.0.

12. The pretreatment solution according to claim 11, wherein in the pretreatment solution, the Tris-HCl is present at a concentration of 10 mM to 200 mM, the EDTA-2Na is present at a concentration of 8 mM to 50 mM, the sodium chloride is present at a concentration of 0.5% to 2% (w/v), the RNase inhibitor is present at a concentration of 2 U/mL to 800 U/mL, and the antibiotic is present at a concentration of 0.005% to 0.05%.

13. The pretreatment solution according to claim 11, wherein in the pretreatment solution, the Tris-HCl is present at a concentration of 80 mM to 120 mM, the EDTA-2Na is present at a concentration of 10 mM to 15 mM, the sodium chloride is present at a concentration of 0.8% to 1% (w/v), and the RNase inhibitor is present at a concentration of 10 U/mL to 30 U/mL.

14. The pretreatment solution according to claim 11, wherein the RNase inhibitor is diethyl pyrocarbonate (DEPC), a RNase protein inhibitor (RNasin), a ribonucleoside vanadyl complex, or SDS; and/or,
the antibiotic is a Proclin antibiotic or NaN₃.

15. The pretreatment solution according to claim 11, wherein the antibiotic is Proclin 300 or Proclin 950.

16. The pretreatment solution according to claim 11, wherein the virus is a DNA virus or an RNA virus.

17. The pretreatment solution according to claim 11, wherein the virus is coronavirus, respiratory syncytial virus, or enterovirus.

18. The pretreatment solution according to claim 11, wherein the virus is SARS-CoV-2.

19. A kit for viral nucleic acid detection based on the one-step method, the kit comprising the pretreatment solution defined according to claim 11.

20. The kit according to claim 19, wherein the kit further comprises a sample releasing agent and a qPCR amplification reagent.
